(19) United States
(12) Patent Application Publication
(10) Pub. No.: US 2025/0036916 A1
(43) Pub. Date: Jan. 30, 2025

(54) PREDICTING WEIGHT CHANGE BASED ON GENETIC INFORMATION AND ACTIVITY INFORMATION

(71) Applicant: FitNow, Inc., New York, NY (US)

(72) Inventors: Gregory SLIWOSKI, Cambridge, MA (US); Edward W. LOWE, JR., Walpole, MA (US)

(21) Appl. No.: 18/361,473

(22) Filed: Jul. 28, 2023

(51) Int. Cl. G06N 3/042; G06N 3/045; G06N 3/0985; G16B 30/00; G16B 40/20; G16H 10/60; G16H 20/60; G16H 50/20

(52) U.S. Cl. CPC G06N 3/042; G06N 3/045; G06N 3/0985; G16B 30/00; G16B 40/20; G16H 10/60; G16H 20/60; G16H 50/20; G16H 50/70

(57) ABSTRACT

A system can train a plurality of machine learning models to predict a weight change. The plurality of machine learning models can include a first model that generates an initial prediction and a second model that generates a final prediction based on the initial prediction. The plurality of machine learning models can be trained based on a collection of historical genetic information, historical activity information, and historical weight information about a plurality of users. The system can receive genetic information and activity information about a new user. The system can then invoke the plurality of machine learning models to predict a weight change of the new user based on the training, the genetic information, and the activity information. Other aspects are also described and claimed.

This disclosure relates generally to predicting weight change and, more specifically, to predicting weight change based on genetic information and activity information. Other aspects are also described.

### BACKGROUND

Various tools exist today to assist individuals with weight loss. For example, in addition to digital scales and traditional exercise equipment, various software tools are also available for assessing foods and exercises, tracking food consumptions, and tracking exercise activities. Further, users can conveniently access such software tools from mobile devices throughout the day. This can enable them to obtain immediate feedback about foods they encounter and enable them to perform immediate logging of foods they consume and exercise they perform.

### SUMMARY

Implementations of this disclosure may provide utilizing ensemble learning to receive a combination of static genetic information (e.g., a DNA sequence) and dynamic activity information (e.g., a dietary consumption log and/or an exercise log) to predict weight change on a continuous basis (e.g., weekly, or monthly intervals). The ensemble learning, using the combination of static genetic information and dynamic activity information as input, may enable a system to indicate user progress toward weight loss with greater accuracy that is specific to the user.

Some implementations may include a method including training a plurality of machine learning models to predict a weight change, the plurality of machine learning models including a first model that generates an initial prediction and a second model that generates a final prediction based on the initial prediction, wherein the plurality of machine learning models is trained based on a collection of historical genetic information, historical activity information, and historical weight information about a plurality of users; receiving genetic information and activity information about a new user; and invoking the plurality of machine learning models to predict a weight change of the new user based on the training, the genetic information, and the activity information.

Some implementations may include an apparatus including a memory and a processor configured to execute instructions stored in the memory to train a plurality of machine learning models to predict a weight change, the plurality of machine learning models including a first model that generates an initial prediction and a second model that generates a final prediction based on the initial prediction, wherein the plurality of machine learning models is trained based on a collection of historical genetic information, historical activity information, and historical weight information about a plurality of users; receive genetic information and activity information about a new user; and invoke the plurality of machine learning models to predict a weight change of the new user based on the training, the genetic information, and the activity information.

Some implementations may include a non-transitory computer readable medium storing instructions operable to cause one or more processors to perform operations including training a plurality of machine learning models to predict a weight change, the plurality of machine learning models including a first model that generates an initial prediction and a second model that generates a final prediction based on the initial prediction, wherein the plurality of machine learning models is trained based on a collection of historical genetic information, historical activity information, and historical weight information about a plurality of users; receiving genetic information and activity information about a new user; and invoking the plurality of machine learning models to predict a weight change of the new user based on the training, the genetic information, and the activity information.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 is a block diagram of an example of a system for predicting weight change based on genetic information and activity information.

FIG. 2 is a block diagram of an example of another system for predicting weight change based on genetic information and activity information.

FIG. 3 is an example of a user device for logging activity information and receiving a predicted weight change.

FIG. 4 is a block diagram of an example internal configuration of a computing device for predicting a weight change based on genetic information and activity information.

FIG. 5 is a flowchart of an example of a process for predicting a weight change based on genetic information and activity information.

### DETAILED DESCRIPTION

Traditional software tools for weight loss may be lacking in their usefulness to users. For example, while some tools may be useful for counting calories and suggesting diet plans, they are often generalized to many different users based on generic classifications, such as age, gender, and/or body mass index (BMI). More recently, some tools have attempted to improve their usefulness to users by further considering a user's specific genetic information. For example, based on a particular user's Deoxyribonucleic acid (DNA) sequence, a system can suggest foods that may be impactful toward weight loss for the user. However, such tools are still limited to the extent that they lack guidance to users. For example, while the information provided by such recent systems may be helpful, a user may nevertheless remain uncertain as to their progress toward weight loss.

Implementations of this disclosure address problems such as these by utilizing ensemble learning that receives a combination of static genetic information and dynamic activity information to predict weight change on a continuous basis. Some implementations may include a system that trains a plurality of machine learning models to predict a weight change. The plurality of machine learning models can include a first model that generates an initial prediction and a second model that generates a final prediction based on the initial prediction. Further, each model of the plurality of machine learning models may be different. For example, each model may be different by one or more hyperparameters. The plurality of machine learning models can be trained based on a collection of historical genetic information (e.g., DNA sequences), historical activity information (e.g., dietary consumption logs and/or exercise logs), and historical weight information (e.g., weigh-ins over time) about a plurality of users. The collection may be obtained from the plurality of users over multiple years. The system can receive genetic information (e.g., a DNA sequence) and activity information (e.g., a dietary consumption log and/or an exercise log) about a new user. The system can then invoke the plurality of machine learning models to predict a weight change of the new user based on the training, the genetic information, and the activity information. As a result, the system can indicate to user progress toward weight loss with greater accuracy specific to the user.

In some implementations, a system can combine genetic variants (e.g., genetic information) with dietary nutrition and exercise behaviors (e.g., activity information) to generate dynamic insights and/or recommendations for increasing weight loss success through machine learning. This may result in improved outcomes when compared to diet and/or behavior modifications alone. In some implementations, the system can receive an input data set of genetic variants based on associations that utilize nutrition, behavior, and outcomes data from an initial population and genome wide associations from a gene sequencing provider. A bagged, stacked, and/or weighted ensemble learning system can then predict outcomes, and highlight dominant behaviors to be adjusted based on the user's current state, to optimize weight loss outcomes for the user. In some implementations, the system can utilize an entirety of the genetic information, as opposed to focusing on genetic traits associated with certain dietary preferences and/or outcomes. The system can combine this genetic information with years of nutrition, behavior, and outcomes data from the population.

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

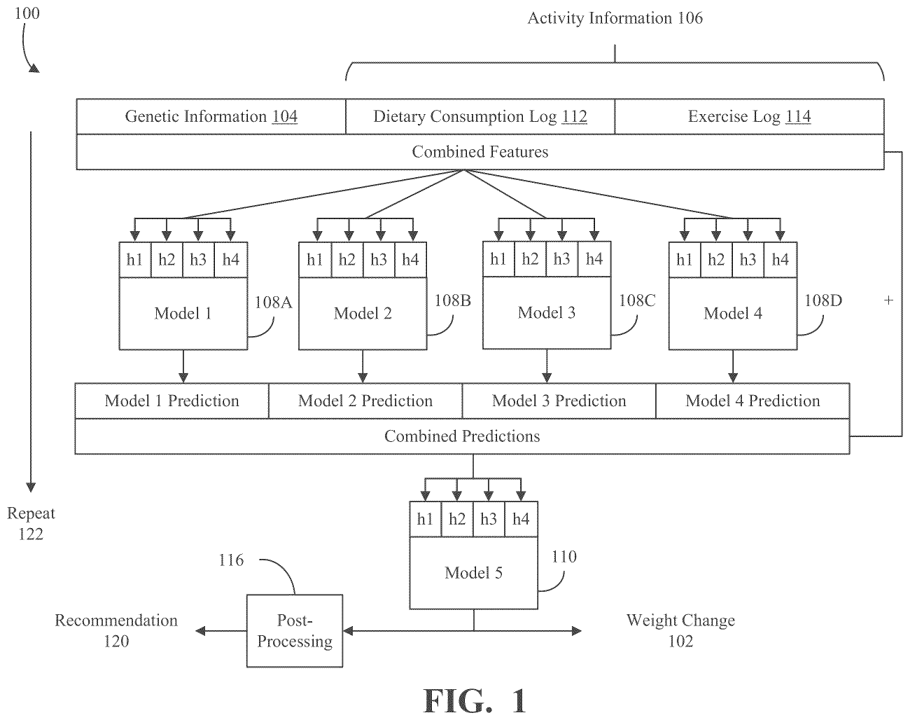

FIG. 1 is a block diagram of an example of a system 100 for predicting a weight change 102 based on genetic information 104 and activity information 106. The system 100 may include a plurality of machine learning models, such as models 108A-108D in a first group and model 110 in a second group. The system 100 may utilize the plurality of machine learning models to predict the weight change 102 of a user based on a combination of static information about the user (e.g., the genetic information 104, which does not change) and dynamic information from the user (e.g., the activity information 106, which may change on a continuous basis).

It has been observed that ensemble learning may provide an advantage when predicting weight changes based on a combination of static information and dynamic information as described herein. Ensemble learning may refer to a technique in which multiple machine learning models are used together to generate a more accurate prediction. The multiple machine learning models may differ from one another, such as by one or more hyperparameters. In some implementations, the multiple machine learning models may include two or more models operating in parallel, such as models 108A and 108B. In some implementations, the multiple machine learning models may include two or more models operating in series or cascaded, such as model 108A and model 110. As shown in FIG. 1, the multiple machine learning models include four models operating in parallel (e.g., models 108A-108D) and an additional model (e.g., model 110) operating in series with, or cascaded with, the four models operating in parallel. The models in the first group (e.g., models 108A-108D) may each generate initial predictions utilized by models in the second group (e.g., model 110). The models in the second group (e.g., model 110, in turn, may generate a final prediction based on the initial predictions (e.g., the weight change 102). For example, the models in the first group (e.g., models 108A-108D) may be bagged models that are stacked with models in the second group (e.g., model 110) to implement ensemble learning.

To implement ensemble learning, each model of the plurality of machine learning models (e.g., models 108A-108D and model 110) may be different from one another. For example, each model could be implemented by a neural network that utilizes hyperparameters h1-h4 where values of one or more of the hyperparameters h1-h4 are unique to that model (e.g., other models do not have the same value for a given hyperparameter). In some cases, values of parameters may be determined via learning. For example, some hyperparameters for a neural network may include a type of optimizer used, number of layers, learning rate, batch size, and transfer function(s) used. In some implementations, other hyperparameter may include a cost or epsilon function of a support vector machine, an extension for regression, and/or a depth and breadth of decision tree.

To make predictions, the plurality of machine learning models (e.g., models 108A-108D and model 110) may be trained using a collection of historical genetic information (e.g., DNA sequences from a gene sequencing provider), historical activity information (e.g., dietary consumption logs and/or exercise logs), and historical weight information (e.g., weigh-ins over time) about a plurality of users. The collection may be obtained from the plurality of users over a relatively longer period, such as multiple years. For example, the collection may include DNA sequences linked to dietary consumption logs, exercise logs, and weigh-ins over multiple years. The DNA sequence may include hundreds of thousands of biomarkers (e.g., 500,000 or more) that are linked to different users. In some implementations, the DNA sequences could be text files comprising locations, identifiers, and indications of variants of biomarkers at the locations.

The collection may comprise a training data set that can enable the machine learning models to learn patterns, such as weight changes in relation to a combination of genetics and activities over time. In some implementations, the plurality of machine learning models may be trained based on a subset of genetic markers in DNA sequences of the plurality of users. For example, the plurality of machine learning models may be trained based on less than 10,000 of 500,000 or more genetic markers in the DNA sequence that are determined to be relevant to user outcomes. The training can be periodic, such as by updating the machine learning model on a discrete time interval basis (e.g., once per week or month), or otherwise. The training data set may derive from multiple users (e.g., thousands of users). The training data set may omit certain data samples that are determined to be outliers, such as weight changes exceeding predefined thresholds or limits within a predefined time interval. The machine learning models may, for example, be or include one or more of a neural network (e.g., a convolutional neural network, recurrent neural network, deep neural network, or other neural network), decision tree, vector machine, Bayesian network, cluster-based system, genetic algorithm, deep learning system separate from a neural network, or other machine learning model.

The system 100 may then receive the genetic information 104 and the activity information 106 about a new user. The genetic information 104 may include a DNA sequence of the new user from a gene sequencing provider. For example, the DNA sequence may include hundreds of thousands of biomarkers (e.g., 500,000 or more) that are linked to the new user. In some implementations, the DNA sequence could be a text file comprising locations, identifiers, and indications of variants of biomarkers at the locations. The activity information 106 may be updated by the new user based on recent activity. In some implementations, the activity information 106 may include a dietary consumption log 112 and/or an exercise log 114 of the new user. The dietary consumption log 112 could indicate food items or nutrients consumed by the new user at specific times (e.g., two eggs or 12 grams of protein at 8:00 a.m. on day 1), or overall nutritional information of each meal (e.g., total calories, including calories from fats, carbohydrates, and proteins). The exercise log 114 could indicate exercises performed by the new user at specific times (e.g., ten pushups at 12:00 p.m. on day 1).

The system 100 may then invoke the plurality of machine learning models to predict the weight change 102 of the new user based on the training, the genetic information 104, and the activity information 106. The system 100 can provide a combination of the genetic information 104 and the activity information 106 as combined features that are input to the models in the first group (e.g., models 108A-108D). The models in the first group can then generate initial predictions based on those combined features (e.g., model 1 prediction from model 108A, model 2 prediction from model 108B, model 3 prediction from model 108C, and model 4 prediction from model 108D). The initial predictions can then be sent as combined predictions to models in the second group (e.g., the model 110). The models in the second group (e.g., model 110) can then generate a final prediction (e.g., the weight change 102) based on the combined predictions and the combined features. As a result, the system 100 can indicate to the new user the weight change 102, which may indicate progress toward weight loss with greater accuracy specific to the new user.

In some implementations, the plurality of machine learning models may predict the weight change 102 based on a subset of genetic markers in the DNA sequence of the new user. For example, the models in the first group (e.g., models 108A-108D) and/or the models in the second group (e.g., the model 110) may generate predictions based on less than 10,000 of 500,000 or more genetic markers in the DNA sequence that are determined to be relevant to user outcomes.

In some implementations, the system 100 can perform post-processing 116 to further generate a recommendation 120 to the new user. For example, the post-processing 116 can determine a portion of the activity information 106 that is a dominant contributor to the weight change 102. The post-processing 116 can then generate a recommendation 120 based on the determined portion. For example, the post-processing 116 can highlight dominant behaviors to be adjusted based on the user's current state. In some implementations, the recommendation 120 may be to increase or decrease consumption of a nutrient for a specified duration. For example, the recommendation 120 could be to increase protein by 10 grams at breakfast on 2 days next week. In some implementations, the recommendation 120 may be to increase or decrease an exercise for a specified duration.

The system 100 can repeat 122 generation of the weight change 102 and/or the recommendation 120 on a continuous basis (e.g., daily, weekly, or monthly). For example, the system 100 can receive an update of the activity information 106 on a weekly basis. The system 100 can provide a combination of the genetic information 104 and update of the activity information 106 as an update of combined features that are input to the models in the first group (e.g., models 108A-108D). The models in the first group can then generate an update of initial predictions based on the update of those combined features (e.g., model 1 prediction from model 108A, model 2 prediction from model 108B, model 3 prediction from model 108C, and model 4 prediction from model 108D). The update of the initial predictions can then be sent as an update of combined predictions to models in the second group (e.g., the model 110). The models in the second group (e.g., model 110) can then generate a final prediction (e.g., an update of the weight change 102) based on the updates of the combined predictions and the combined features.

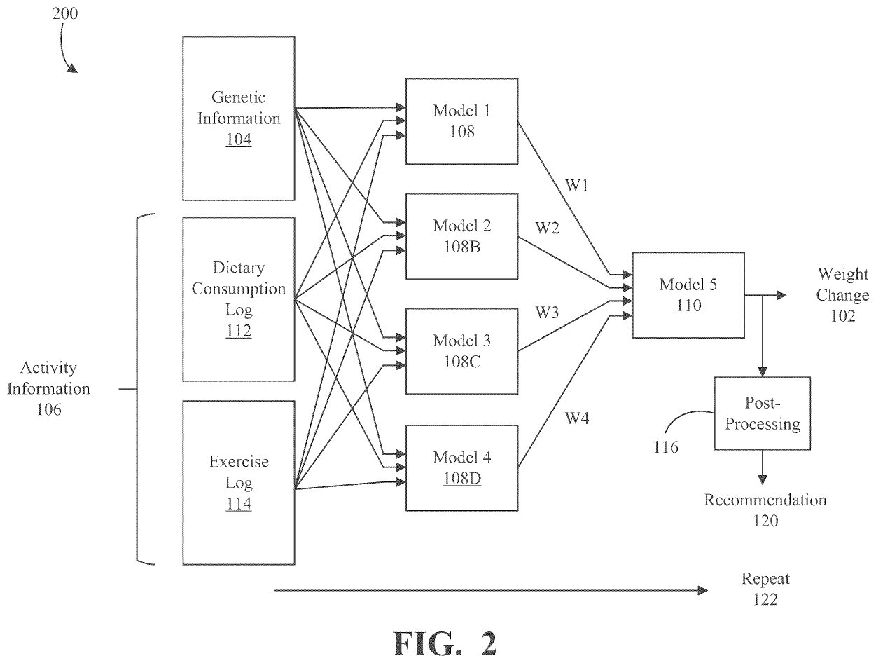

FIG. 2 is a block diagram of an example of a system 200 for predicting weight change based on genetic information and activity information. The system 200 may be like the system 100 where like reference numerals refer to corresponding parts throughout. In the system 200, the plurality of machine learning models may be weighted. For example, initial predictions generated by the models in the first group may be weighted, such as model 1 prediction from model 108A having a weight W1, model 2 prediction from model 108B having a weight W2, model 3 prediction from model 108C having a weight W3, and model 4 prediction from model 108D having a weight W4. For example, the system 200 could be a bagged, stacked, and weighted ensemble in which weighted predictions are used to generate a final prediction (e.g., the weight change 102). In some implementations, the weighting may be determined based on machine learning,

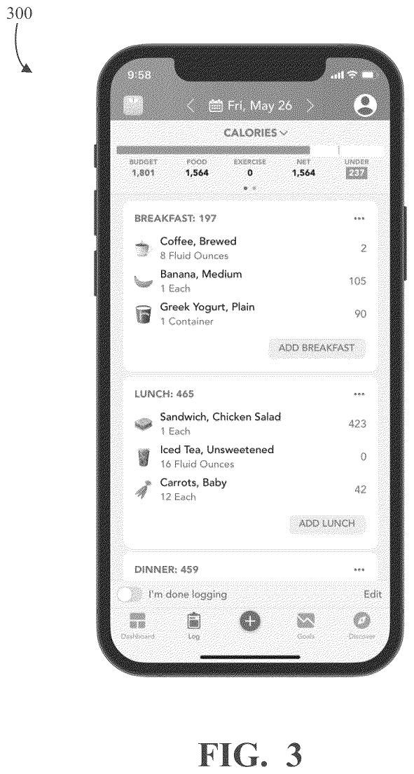

FIG. 3 is an example of a user device 300 for logging the activity information 106 and receiving a predicted weight change (e.g., the weight change 102). The user device 300 may be a mobile device such as smartphone, tablet, or laptop. The user device 300 may include a display configured to output a graphical user interface (GUI) to enable the user to generate an account, link their genetic information 104 (e.g., from a gene sequencing provider), and enter their activity information 106 on a continuous basis. The GUI may further enable the user to receive the predicted weight change, based on the genetic information 104 that is linked and the activity information 106 that is entered, on a continuous basis (e.g., daily, weekly, or monthly).

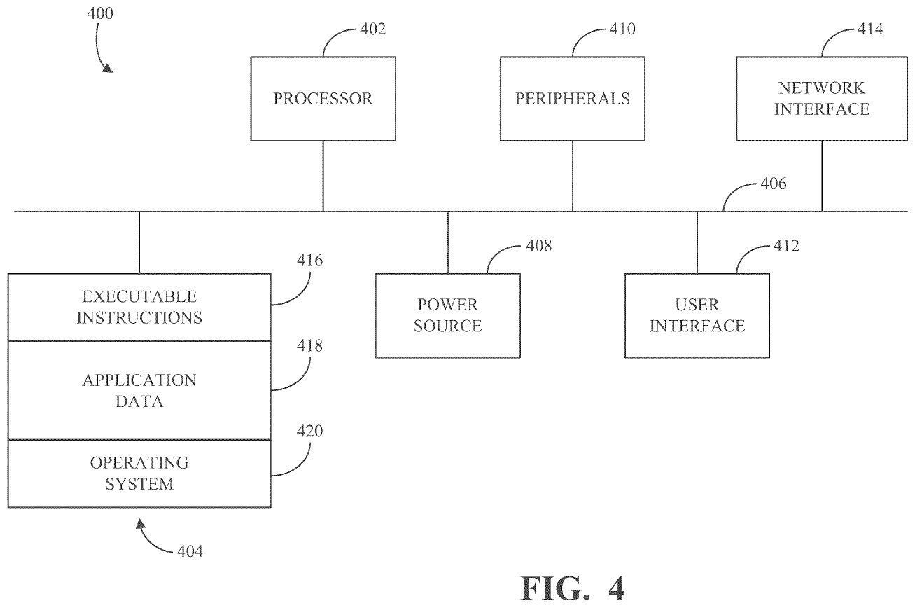

FIG. 4 is a block diagram of an example internal configuration of a computing device 400 for predicting a weight change based on genetic information and activity information. In one configuration, the computing device 400 may comprise an apparatus configured to predict weight changes based on genetic information and activity information. For example, the computing device 400 may implement the system 100 and/or the system 200. In another configuration, the computing device 400 may comprise a user device for logging activity information and receiving a predicted weight change. For example, the computing device 400 may implement the user device 300.

The computing device 400 includes components or units, such as a processor 402, a memory 404, a bus 406, a power source 408, peripherals 410, a user interface 412, a network interface 414, other suitable components, or a combination thereof. One or more of the memory 404, the power source 408, the peripherals 410, the user interface 412, or the network interface 414 can communicate with the processor 402 via the bus 406.

The processor 402 is a central processing unit, such as a microprocessor, and can include single or multiple processors having single or multiple processing cores. Alternatively, the processor 402 can include another type of device, or multiple devices, configured for manipulating or processing information. For example, the processor 402 can include multiple processors interconnected in one or more manners, including hardwired or networked. The operations of the processor 402 can be distributed across multiple devices or units that can be coupled directly or across a local area or other suitable type of network. The processor 402 can include a cache, or cache memory, for local storage of operating data or instructions.

The memory 404 includes one or more memory components, which may each be volatile memory or non-volatile memory. For example, the volatile memory can be random access memory (RAM) (e.g., a DRAM module, such as DDR DRAM). In another example, the non-volatile memory of the memory 404 can be a disk drive, a solid state drive, flash memory, or phase-change memory. In some implementations, the memory 404 can be distributed across multiple devices. For example, the memory 404 can include network-based memory or memory in multiple clients or servers performing the operations of those multiple devices.

The memory 404 can include data for immediate access by the processor 402. For example, the memory 404 can include executable instructions 416, application data 418, and an operating system 420. The executable instructions 416 can include one or more application programs, which can be loaded or copied, in whole or in part, from non-volatile memory to volatile memory to be executed by the processor 402. For example, the executable instructions 416 can include instructions for performing some or all of the techniques of this disclosure. The application data 418 can include user data, database data (e.g., database catalogs or dictionaries), or the like. In some implementations, the application data 418 can include functional programs, such as a web browser, a web server, a database server, another program, or a combination thereof. The operating system 420 can be, for example, Microsoft Windows®, Mac OS X®, or Linux®; an operating system for a mobile device, such as a smartphone or tablet device; or an operating system for a non-mobile device, such as a mainframe computer.

The power source 408 provides power to the computing device 400. For example, the power source 408 can be an interface to an external power distribution system. In another example, the power source 408 can be a battery, such as where the computing device 400 is a mobile device or is otherwise configured to operate independently of an external power distribution system. In some implementations, the computing device 400 may include or otherwise use multiple power sources. In some such implementations, the power source 408 can be a backup battery.

The peripherals 410 includes one or more sensors, detectors, or other devices configured for monitoring the computing device 400 or the environment around the computing device 400. For example, the peripherals 410 can include a geolocation component, such as a global positioning system location unit. In another example, the peripherals can include a temperature sensor for measuring temperatures of components of the computing device 400, such as the processor 402. In some implementations, the computing device 400 can omit the peripherals 410.

The user interface 412 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, virtual reality display, or other suitable display.

The network interface 414 provides a connection or link to a network. The network interface 414 can be a wired network interface or a wireless network interface. The computing device 400 can communicate with other devices via the network interface 414 using one or more network protocols, such as using Ethernet, transmission control protocol (TCP), internet protocol (IP), power line communication, an IEEE 802.X protocol (e.g., Wi-Fi, Bluetooth, or ZigBee), infrared, visible light, general packet radio service (GPRS), global system for mobile communications (GSM), code-division multiple access (CDMA), Z-Wave, another protocol, or a combination thereof.

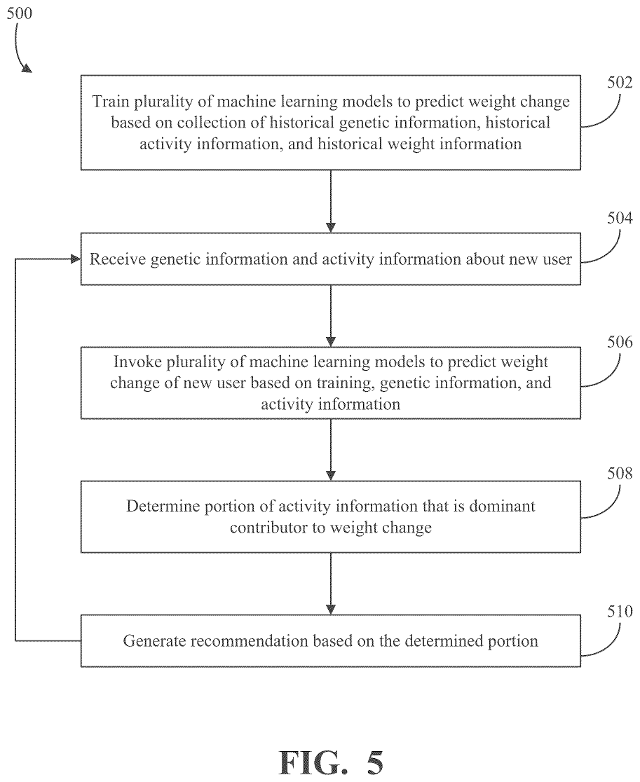

FIG. 5 is a flowchart of an example of a process 500 for predicting a weight change based on genetic information and activity information. The process 500 can be executed using computing devices, such as the systems, hardware, and software described with respect to FIGS. 1-4. The process 500 can be performed, for example, by executing a machine-readable program or other computer-executable instructions, such as routines, instructions, programs, or other code. The operations of the process 500 or another technique, method, process, or algorithm described in connection with the implementations disclosed herein can be implemented directly in hardware, firmware, software executed by hardware, circuitry, or a combination thereof.

For simplicity of explanation, the process 500 is depicted and described herein as a series of operations. However, the operations in accordance with this disclosure can occur in various orders and/or concurrently. Additionally, other operations not presented and described herein may be used. Furthermore, not all illustrated operations may be required to implement a technique in accordance with the disclosed subject matter.

At operation 502, a system may train a plurality of machine learning models to predict a weight change. The plurality of machine learning models may include a first model that generates an initial prediction and a second model that generates a final prediction based on the initial prediction. For example, the plurality of machine learning models may include models 108A-108D in a first group and model 110 in a second group. The models 108A-108D in the first group may generate initial predictions, and model 110 in the second group may generate a final prediction based on the initial prediction. The plurality of machine learning models may be trained based on a collection of historical genetic information, historical activity information, and historical weight information about a plurality of users. The collection may be obtained from a plurality of users over a relatively longer period, such as multiple years.

At operation 504, the system may receive genetic information and activity information about a new user. For example, the system may receive the genetic information 104 and the activity information 106 about the new user. The genetic information may include a DNA sequence of the new user. The activity information may include a dietary consumption log and/or an exercise log of the new user. The new user could use the user device 300 to provide the genetic information and the activity information to the system.

At operation 506, the system may invoke the plurality of machine learning models to predict a weight change of the new user based on the training, the genetic information, and the activity information. For example, the system can provide a combination of the genetic information and the activity information of the new user as combined features that are input to models in the first group (e.g., models 108A-108D). The models in the first group can then generate initial predictions based on those combined features. The initial predictions can then be sent as combined predictions to models in the second group (e.g., model 110). The models in the second group can then generate a final prediction (e.g., the weight change 102) based on the combined predictions and the combined features.

At operation 508, the system may determine a portion of the activity information that is a dominant contributor to the weight change. For example, the system can perform post-processing (e.g., the post-processing 116) to determine a portion of the activity information that is a dominant contributor to the weight change.

At operation 510, the system may generate a recommendation (e.g., the recommendation 120) based on the determined portion. For example, the recommendation may be to increase or decrease consumption of a nutrient for a specified duration. In another example, the recommendation may be to increase or decrease an exercise for a specified duration.

In utilizing the various aspects of the embodiments, it would become apparent to one skilled in the art that combinations or variations of the above embodiments are possible for forming a fan out system in package including multiple redistribution layers. Although the embodiments have been described in language specific to structural features and/or methodological acts, it is to be understood that the appended claims are not necessarily limited to the specific features or acts described. The specific features and acts disclosed are instead to be understood as embodiments of the claims useful for illustration.

What is claimed is:

1. A method, comprising:
training a plurality of machine learning models to predict a weight change, the plurality of machine learning models including a first model that generates an initial prediction and a second model that generates a final prediction based on the initial prediction, wherein the plurality of machine learning models is trained based on a collection of historical genetic information, historical activity information, and historical weight information about a plurality of users;
receiving genetic information and activity information about a new user; and
invoking the plurality of machine learning models to predict a weight change of the new user based on the training, the genetic information, and the activity information.

2. The method of claim 1, wherein the plurality of machine learning models comprises a first group of two or more models, including the first model, that generates two or more initial predictions utilized by the second model.

3. The method of claim 1, wherein the plurality of machine learning models utilize ensemble learning to predict the weight change.

4. The method of claim 1, wherein each model of the plurality of machine learning models differs by at least one hyperparameter.

5. The method of claim 1, further comprising:
determining a portion of the activity information that is a dominant contributor to the weight change; and
generating a recommendation based on the determined portion.

6. The method of claim 5, wherein the recommendation is to increase or decrease consumption of a nutrient for a specified duration.

7. The method of claim 1, wherein the activity information includes a dietary consumption log of the new user.

8. The method of claim 1, wherein the activity information includes an exercise log of the new user.

9. The method of claim 1, wherein the weight change is predicted based on a weekly interval of the activity information.

10. The method of claim 1, wherein the plurality of machine learning models predicts the weight change based on a subset of genetic markers in a Deoxyribonucleic acid (DNA) sequence of the new user.

11. The method of claim 1, wherein the collection is from the plurality of users over multiple years.

12. An apparatus, comprising:
a memory; and
a processor configured to execute instructions stored in the memory to:
train a plurality of machine learning models to predict a weight change, the plurality of machine learning models including a first model that generates an initial prediction and a second model that generates a final prediction based on the initial prediction, wherein the plurality of machine learning models is trained based on a collection of historical genetic information, historical activity information, and historical weight information about a plurality of users;
receive genetic information and activity information about a new user; and
invoke the plurality of machine learning models to predict a weight change of the new user based on the training, the genetic information, and the activity information.

13. The apparatus of claim 12, wherein the plurality of machine learning models comprises two or more bagged models, including the first model, that generate two or more initial predictions utilized by the second model.

14. The apparatus of claim 12, wherein the processor is further configured to execute instructions stored in the memory to:
determine a food item that is a dominant contributor to the weight change; and
generate a recommendation based on the food item or nutrient.

15. The apparatus of claim 12, wherein the processor is further configured to execute instructions stored in the memory to:
determine an exercise that is a dominant contributor to the weight change; and
generate a recommendation based on the exercise.

16. The apparatus of claim 12, wherein the weight change is predicted based on a monthly interval of the activity information.

17. A non-transitory computer readable medium storing instructions operable to cause one or more processors to perform operations comprising:
training a plurality of machine learning models to predict a weight change, the plurality of machine learning models including a first model that generates an initial prediction and a second model that generates a final prediction based on the initial prediction, wherein the plurality of machine learning models is trained based on a collection of historical genetic information, historical activity information, and historical weight information about a plurality of users;
receiving genetic information and activity information about a new user; and
invoking the plurality of machine learning models to predict a weight change of the new user based on the training, the genetic information, and the activity information.

18. The non-transitory computer readable medium storing instructions of claim 17, wherein the plurality of machine learning models comprises the first model and another model that each generate initial predictions utilized by the second model.

19. The non-transitory computer readable medium storing instructions of claim 17, wherein each model of the plurality of machine learning models is implemented by a neural network.

20. The non-transitory computer readable medium storing instructions of claim 17, wherein the activity information is a combination of dietary consumption information and exercise information.
